(19) United States
(12) Patent Application Publication
(10) Pub. No.: US 2026/0014333 A1
(43) Pub. Date: Jan. 15, 2026

(54) SYSTEM AND METHODS FOR CONTINUOUSLY MONITORING A CONCENTRATION OF VOLATILE BREATHING COMPOUNDS

(71) Applicant: Masimo Corporation, Irvine, CA (US)

(72) Inventors: Per FERNKVIST, Sollentuna (SE); Hithesh Kumar GATTY, Uppsala (SE)

(21) Appl. No.: 19/258,030

(22) Filed: Jul. 2, 2025

Related U.S. Application Data
(60) Provisional application No. 63/669,631, filed on Jul. 10, 2024.
(60) Provisional application No. 63/669,635, filed on Jul. 10, 2024.

(51) Int. Cl. A61M 16/00; A61M 16/10; A61M 16/12; G01N 27/30; G01N 27/413

(52) U.S. Cl. CPC A61M 16/0003; A61M 16/024; A61M 16/104; A61M 16/12; G01N 27/308; G01N 27/413; A61M 2202/0275; A61M 2205/3317; A61M 2230/432

(57) ABSTRACT

Disclosed herein are systems and methods for monitoring at least one volatile breathing compound in respiratory gases of patients. A device includes a patient respiratory gas interface coupled via a connector to a gas sampling line. The line includes a first channel for conducting a predetermined flux of respiratory gases from the interface to an inlet of a sidestream gas monitor, which monitors carbon dioxide (CO₂) content. A pump facilitates gas flow through the device and the monitor. An outlet of the monitor is coupled to a second channel for conveying gases away. A valve controls passage of respiratory gas from the monitor via the second channel by connecting or disconnecting the flux to/from at least one volatile breathing compound detector, which monitors compound content. A control unit monitors CO₂ values and uses this as a signal to control the valve, enabling compound monitoring or bypass based on CO₂ content.

### RELATED APPLICATIONS

Any and all applications for which a foreign or domestic priority claim is identified in the Application Data Sheet as filed with the present application are hereby incorporated by reference under 37 CFR 1.57.

This application claims the priority benefit of U.S. Provisional Patent Application 63/669,631 filed on Jul. 10, 2024, entitled “A DEVICE, A METHOD AND A SYSTEM FOR CONTINUOUS MONITORING A CONCENTRATION OF VOLATILE BREATHING COMPOUNDS,” and U.S. Provisional Patent Application 63/669,635 filed on July 10, entitled “MINIATURISED ELECTROCHEMICAL SENSOR, DEVICE AND METHOD OF MANUFACTURING,” which are incorporated by reference herein in their entirety.

### BACKGROUND

### Field

This disclosure relates to a device, a method, and a system for continuous monitoring of a concentration of at least one volatile breathing compound in respiratory gases of patients, which patients may either be intubated or self-breathing. More specifically, the disclosure relates to a continuous monitoring of carbon monoxide and/or nitric oxide in respiratory gases.

### Related Art

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

The content and concentration of certain respiratory gases of patients (e.g., their exhaled breath) can reveal physiological information about a person, such as a potential presence of inflammatory diseases in the lungs. Several components of the respiratory gases are either produced or altered by the cells of the lungs and the respiratory tract. The physiological information that can be examined may for instance be used to diagnose pathological conditions and/or the effect of a particular treatment. Several volatile breathing compounds exist that may and/or have been shown to reveal physiological information. Two of these indicative components are nitric oxide (NO) and carbon monoxide (CO) even though many other components may convey indications of other respiratory conditions.

### SUMMARY

For purposes of summarizing the disclosure and the advantages achieved over the prior art, certain objects and advantages of the disclosure are described herein. Not all such objects or advantages may be achieved in any particular embodiment. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these implementations are intended to be within the scope of the invention herein disclosed. These and other implementations will become readily apparent to those skilled in the art from the following detailed description of the preferred implementations having reference to the attached figures.

In some implementations, a device for continuous monitoring of a concentration of at least one volatile breathing compound in respiratory gases of patients can include: a patient respiratory gas interface; a connector adapted to couple the patient respiratory gas interface to a gas sampling line, the gas sampling line including a first channel for conducting a predetermined flux of the respiratory gases from the patient respiratory gas interface to an inlet of a sidestream respiratory gas monitor, wherein the sidestream respiratory gas monitor is adapted to continuously monitor carbon dioxide (CO2) content of the respiratory gases; a pump arranged to facilitate a continuous flux of the respiratory gases through the device including through the sidestream respiratory gas monitor; and an outlet of the sidestream respiratory gas monitor coupled to a second channel of the gas sampling line for conveying the respiratory gases from the sidestream respiratory gas monitor, the second channel coupled to a valve, wherein the valve is adapted to control passage of the respiratory gas from the sidestream respiratory gas monitor via the second channel by connecting or disconnecting the gas flux to or from at least one volatile breathing compound detector, wherein the at least one volatile breathing compound detector is adapted to continuously monitor volatile breathing compound content of the respiratory gases led to the at least one breathing compound via the valve; wherein a momentary value of the continuously monitored CO2 content of the respiratory gases is monitored by a control unit, wherein the control unit is configured to transmit a signal to the valve based on the momentary value, wherein the passage of respiratory gas though the valve is controlled in dependence of the signal, wherein a provision of respiratory gas can be controlled to be either lead through the at least one volatile breathing compound detector or by-passed in dependence on the momentary value of the CO2 content.

In some implementations, the pump is located downstream of the sidestream respiratory gas monitor and downstream of the valve and volatile breathing compound detector, wherein the pump acts on the respiratory gases irrespective of whether the respiratory gases are led through at least one volatile breathing compound detector or by-passed. In some implementations, the volatile breathing compound includes at least one of carbon monoxide (CO) and nitric oxide (NO). In some implementations, the predetermined flux of the respiratory gases includes 30-70 ml/min. In some implementations, the predetermined flux of the respiratory gases includes 40-60 ml/min. In some implementations, the predetermined flux of the respiratory gases includes 50 ml/min.

In some implementations, the device is adapted to function in a manner such that a CO2 controlled signal indicating that 70% to 90% of a patient's breath is exhaled causes the control unit controls to control the valve to allow the respiratory gases from the second channel to enter into an area of the at least one volatile breathing compound detector, and that the CO2 controlled signal indicates that a portion of or all of a patent's breath is exhaled causes the control unit control to control the valve to by-pass the respiratory gases from the second channel as exhaust gases. In some implementations, the control unit allows the respiratory gases from the second channel to enter the at least one volatile breathing compound detector once 80% of a patient's breath is exhaled.

In some implementations, the at least one volatile breathing compound detector is slower in response time in relation to a patient's breathing cycles. In some implementations, the at least one volatile breathing compound detector includes an electrochemical sensor detecting at least one of CO and NO. In some implementations, the at least one volatile breathing compound detector has a response rate of up to 90% of a response value in approximately 30 seconds.

In some implementations, the device is adapted for intubated patients. In some implementations, the device is adapted for spontaneously or self-breathing patients in that particularly high concentrations of CO and/or NO are measured.

In some implementations, a method for continuous monitoring of a concentration of at least one volatile breathing compound in respiratory gases of patients can include: coupling a patient respiratory gas interface to a gas sampling line and a sidestream respiratory gas monitor, wherein the sidestream respiratory gas monitor continuously monitoring carbon dioxide (CO2) content of the respiratory gases, and wherein a pump is facilitating a continuous flux of the respiratory gases through the sidestream respiratory gas monitor; actuating a valve controlling passage of the respiratory gas from the sidestream respiratory gas monitor by connecting or disconnecting the gas flux to or from at least one volatile breathing compound detector which continuously monitors volatile breathing compound content of the respiratory gases, and utilizing a momentary value of the continuously monitored CO2 content of the respiratory gases monitored by a control unit as a signal to the valve, so as to control passage of respiratory gas though the valve in dependence of the signal.

In some implementations, the pump is located downstream of the sidestream respiratory gas monitor and downstream of the valve and the at least one volatile breathing compound detector, and wherein the pump acts on the respiratory gases irrespective of whether the respiratory gases are led through the at least one volatile breathing compound detector or bypassed. In some implementations, the at least one volatile breathing compound includes at least one of carbon monoxide (CO) and nitric oxide (NO). In some implementations, the gas flux of the respiratory gases through the sidestream respiratory gas monitor includes 30-70 ml/min. In some implementations, the gas flux of the respiratory gases through the sidestream respiratory gas monitor includes 40-60 ml/min. In some implementations, the gas flux of the respiratory gases through the sidestream respiratory gas monitor includes about 50 ml/min.

In some implementations, the method includes controlling the valve such that a CO2-controlled signal indicating that 70% to 90% of a patient's breath is exhaled causes the control unit to allow the respiratory gases from a second channel to enter an area of the at least one volatile breathing compound detector, and wherein the CO2-controlled signal indicating all of the patient's breath is exhaled causes the control unit to control the valve to bypass the respiratory gases from the second channel as exhaust gases. In some implementations, the control unit allows the respiratory gases from the second channel to enter the at least one volatile breathing compound detector once 80% of the patient's breath is exhaled. In some implementations, the at least one volatile breathing compound detector has a slower response time relative to a patient's breathing cycles. In some implementations, the at least one volatile breathing compound detector includes an electrochemical sensor configured to detect at least one of CO and NO. In some implementations, the at least one volatile breathing compound detector has a response rate up to 90% of a response value in approximately 30 seconds.

In some implementations, a miniaturized electrochemical sensor for detection of a component in a gas can include: a casing including a rigid material; at least two electrodes, one of which includes a working electrode and one of which electrodes includes a counter electrode, wherein both electrodes are at least partly enclosed within the casing while still in contact with an environment surrounding the casing; connection wires connected to each electrode; the casing encapsulating a liquid electrolyte, wherein the liquid electrolyte is contained in-between the working electrode and the counter electrode; and a permeable electrolyte absorbing material provided between the working electrode and the counter electrode, the permeable electrolyte absorbing material including a structure with a plurality of passages extending between the working electrode and the counter electrode to allow ions of the electrolyte to be transported between the electrodes. In some implementations, the miniaturized electrochemical sensor includes a reference electrode provided between the working electrode and the counter electrode and in electrical contact with the electrolyte.

In some implementations, the rigid material casing is formed by sandwiching at least two slab formed structures that are configured to fixedly enclose by fasteners the miniaturized electrochemical sensor and encapsulate the liquid electrolyte. In some implementations, the rigid material casing is 3D-printed. In some implementations, surfaces that face the electrolyte of at least one of the working electrodes and the counter electrodes are divided by filters. In some implementations, wherein the filters include Zitex.

In some implementations, surfaces that face the electrolyte of at least one of the working electrodes and the counter electrodes are divided by an electrolyte absorbing filter material. In some implementations, the electrolyte absorbing filter material includes POREX®. In some implementations, wherein the structure of the electrolyte absorbing material is porous, wherein the passages are formed as pores. In some implementations, the pores of the electrolyte absorbing material extend in parallel throughout the structure of the electrolyte absorbing material.

In some implementations, a surface of the working electrode facing the gas includes at least one of gold, palladium, ruthenium, and/or platinum coated and the surface facing the electrolyte is coated and/or covered by carbon. In some implementations, at least one connection wire connected to an electrode is comprised from at least one of gold, palladium, ruthenium, and/or platinum. In some implementations, the miniaturized electrochemical sensor is configured for measuring content of CO in exhaled breath.

In some implementations, a method of manufacturing a miniaturized electrochemical sensor device for detection of a component in a gas can include: providing a casing made from rigid material, providing at least two electrodes, one of which electrodes is a working electrode and one of which electrodes is a counter electrode, wherein both electrodes are at least partly enclosed within the casing while still in contact with an environment surrounding the casing, connecting connection wires to each electrode, enclosing a liquid electrolyte with the casing, wherein the liquid electrolyte is contained in-between the working electrode and the counter electrode; and providing a permeable electrolyte absorbing material between the working electrode and the counter electrode, the permeable electrolyte absorbing material including a structure with a plurality of passages extending between the working electrode and the counter electrode to allow ions of the electrolyte to be transported between the electrodes.

In some implementations, the liquid electrolyte includes sulfuric acid (H2SO4). In some implementations, the method includes: providing a reference electrode between the working electrode and the counter electrode and in electrical contact with the electrolyte. In some implementations, the method includes forming the rigid material casing by sandwiching at least two slab formed structures that are configured to fixedly enclose by fasteners the sensor device and encapsulate the liquid electrolyte. In some implementations, the rigid material casing is 3D-printed.

In some implementations, surfaces that face the electrolyte of at least one of the working electrodes and the counter electrodes are divided by filters. In some implementations, the filters include Zitex. In some implementations, surfaces that face the electrolyte of at least one of the working electrodes and the counter electrodes are divided by an electrolyte absorbing filter material.

In some implementations, the electrolyte absorbing filter material includes POREX®. In some implementations, the structure of the electrolyte absorbing material is porous, wherein the passages are formed as pores. In some implementations, the pores of the electrolyte absorbing material extend in parallel throughout the structure of the electrolyte absorbing material.

In some implementations, a surface of the working electrode facing the gas includes at least one of gold, palladium, ruthenium, and/or platinum coated and the surface facing the electrolyte is coated and/or covered by carbon. In some implementations, at least one connection wire connected to an electrode is comprised from at least one of gold, palladium, ruthenium, and/or platinum. In some implementations, the miniaturized electrochemical sensor is configured for measuring content of CO in exhaled breath.

### BRIEF DESCRIPTION OF THE DRAWINGS

Various implementations will be described hereinafter with reference to the accompanying drawings. These implementations are illustrated and described by example only and are not intended to limit the scope of the disclosure. In the drawings, similar elements have similar reference numerals. It is to be understood that the accompanying drawings, which are incorporated in and constitute a part of this specification, are for the purpose of illustrating concepts disclosed herein and may not be to scale.

FIG. 1 is a schematic drawing of the structural features of a device for continuous monitoring of a concentration of at least one volatile breathing compound in respiratory gases, according to various implementations of the present disclosure.

FIG. 2 is a schematic diagram of a process for continuously monitoring a concentration of at least one volatile breathing compound in respiratory gases, according to various implementations of the present disclosure.

FIG. 3 shows a perspective explosion view of a miniaturized electrochemical sensor, in which the distance between various components have been extended, according to various implementations of the present disclosure.

FIG. 4 shows side sectional view of the miniaturized electrochemical sensor of FIG. 3, according to various implementations of the present disclosure.

FIGS. 5A-5D show various configurations of a layer of porous polymeric material of the miniaturized electrochemical sensor of FIG. 3, according to various implementations of the present disclosure.

### DETAILED DESCRIPTION

Although several implementations, examples, and illustrations are disclosed below, it will be understood by those of ordinary skill in the art that the devices, systems, and methods described herein extend beyond the specifically disclosed implementations, examples, and illustrations and includes other uses of the devices, systems, and methods and obvious modifications and equivalents thereof. Implementations are described with reference to the accompanying figures, wherein like numerals refer to like elements throughout. The terminology used in the description presented herein is not intended to be interpreted in any limited or restrictive manner simply because it is being used in conjunction with a detailed description of some specific implementations of the devices, systems, and methods. In addition, implementations can comprise several novel features. No single feature is solely responsible for its desirable attributes or is essential to practicing the devices, systems, and methods herein described.

The present disclosure may be understood by reference to the following detailed description. It is noted that, for purposes of illustrative clarity, certain elements in various drawings may not be drawn to scale, may be represented schematically or conceptually, or otherwise may not correspond exactly to certain physical configurations of implementations.

To monitor the concentration of volatile breathing compounds is a relatively new technique in clinical medicine. This technique is based on the discovery that volatile breathing compounds that are exhaled by patients can be measured, and those measurements can provide information on the patient's metabolic state, infection status and other physiological processes. Some of the volatile breathing compounds that are most commonly monitored include carbon monoxide, nitric oxide, and various organic compounds.

The monitoring of volatile breathing compounds can be accomplished through a variety of measurement methods and techniques. One of the existing techniques is breath analysis using gas chromatography-mass spectrometry. This method separates the volatile breathing compounds in exhaled breaths and identifies them based on their molecular weight and characteristic fragmentation pattern. Another existing technique is breath analysis using ion mobility spectrometry. This technique separates volatile breathing compounds in exhaled breaths based on their mobility and ability to be deflected in an electric field and provides a rapid analysis of the breath composition. Other techniques, such as techniques based on the so-called electronic nose, uses a sensor array to detect and identify volatile breathing compounds in exhaled breaths. The electronic nose can distinguish between different patterns of volatile compounds and can identify specific breath signatures. Infrared spectroscopy techniques on the other hand measure the absorption of infrared radiation by exhaled breaths and can identify specific volatile breathing compounds based on analysis of their absorption spectra. All the mentioned techniques entail both advantages and disadvantages and may be suitable for use depending on the circumstances and specific measurement needs.

Common for the various existing techniques presently available for monitoring of volatile breathing compounds is that the monitoring is often the basis that provides valuable information on a patient's physiological status. Another common factor is that the techniques may further aid in the diagnosis and management of various diseases. Further research will more clearly establish the clinical utility of the mentioned techniques. One of several areas in which scientific journals still indicate room for further development is the preparation of standardized protocols for use together with the existing methods and techniques in clinical practice.

While continuous monitoring of a concentration of volatile breathing compounds in respiratory gases, as previously mentioned, has the potential to provide valuable information on the patient's metabolic state and clinical condition, there are several known difficulties associated with this technique. This applies for both intubated patients and patients that are spontaneous or self-breathing. One of these difficulties is variation in breath sampling. This variation refers to the fact that both the quality and quantity of the exhaled breath sample can vary, sometimes significantly, due to factors such as patient effort, respiratory rate, and/or dead space ventilation. Such variability can affect the accuracy and reproducibility of the measurements. Another challenge may arise from limitations in the sensitivity and selectivity of the monitoring device, caused by factors such as interference from other gases, background noise, and instrumental drift. These limitations can impact the accuracy and precision of the measurements. Calibration and maintenance demands of the monitoring device may further require frequent calibration and maintenance actions to ensure accurate and reliable measurements. This requirement is presumably even more challenging in a busy intensive care unit (ICU) environment where technical equipment is used to its maximum, patients typically are severely ill and staff resources often is a limiting factor. Other physiological circumstances, such as the patient's clinical condition, medications, and medical interventions may of course also affect the concentration of volatile breathing compounds in respiratory gases. Such factors may cause further difficulties in obtaining and interpreting results as well as in applying them to clinical decision-making.

Monitoring of respiratory gases in intubated patients is an essential aspect of critical care management. There are several ways to monitor respiratory gases in intubated patients, such as the end-tidal carbon dioxide (ETCO₂) monitoring, which is a commonly used non-invasive method of monitoring carbon dioxide levels in exhaled breaths. This method can include attaching a CO₂ sensor to the endotracheal tube or the ventilator circuit. ETCO₂ monitoring provides information on ventilation, cardiac output and metabolic status. Continuous monitoring of respiratory gases in intubated patients helps clinicians to adjust ventilation parameters and to manage the patient's condition effectively. As previously mentioned, it is important to monitor these gases to ensure adequate oxygenation and ventilation, prevent complications, and optimize patient outcomes. Overall, while the continuous monitoring of volatile breathing compounds in respiratory gases of both self-breathing and intubated patients is a promising technique, as has been previously mentioned, there are several challenges that need to be addressed to enable its widespread clinical use.

In view of the above, there is a need for an improved device for continuous monitoring of a concentration of at least one volatile breathing compound in respiratory gases of both self-breathing and intubated patients. There is also a need for corresponding inventive methods and systems that may supplement the performance and usability of the device.

Additionally, the content and concentration of respiratory gases in breaths of patients, the respiratory gases also known as volatile breathing compounds, can be used as indicators of pulmonary conditions. Furthermore, the content and concentration of fractional exhaled nitric oxide and exhaled carbon monoxide have been evaluated as indicators of inflammatory pulmonary diseases, such as asthma and related chronic obstructive pulmonary disease for several decades. In recent years, there has been extensive academic and industrial research particularly on carbon monoxide as a biomarker. One such use of the biomarker is in the mentioned detection of respiratory inflammation, such as asthma. The concentration of carbon monoxide (CO) in exhaled breath serves as a marker of the inflammation in the airways of asthma patients. Thus, the use of exhaled CO (eCO) is considered as a promising tool in diagnosing asthma. However, the concentration of eCO in exhaled breath is relatively low. In a healthy adult, the concentration of eCO is typically less than 10 ppm. Patients with a bacterial infection in the lower respiratory tract go from less than 10 ppm to typically 2-6 ppm after treatment with antibiotics. It is roughly the same values as for other patient groups which show that healthy people are roughly in the range of 1-5 ppm while patents with a bacterial infection are typically in the range of 3-10 ppm.

Light smokers display a concentration of about 10 ppm eCO while heavy smokers can be up to 30 ppm. One therefore ideally wants to be able to measure in the range 0-30 ppm to be able to include also the group of smokers and heavy smokers as well. However, it is difficult to draw other conclusions for the group of smokers than the fact that they smoke. Also, for individuals with considerable amount of respiratory inflammation it is not uncommon to display a much higher concentration by a factor 10 or even more.

Conventional sensors using electrochemical methods can detect CO. However, for measurements of exhaled CO, these sensors lack fast response, small size, and/or high sensitivity. Conventional electrochemical sensors according to the prior art can detect gas concentration down to some parts per billion but they suffer from long response times, typically in the order of 60-100 seconds. Consequently, they require overly complicated flow handling and buffering of the exhaled breath sample.

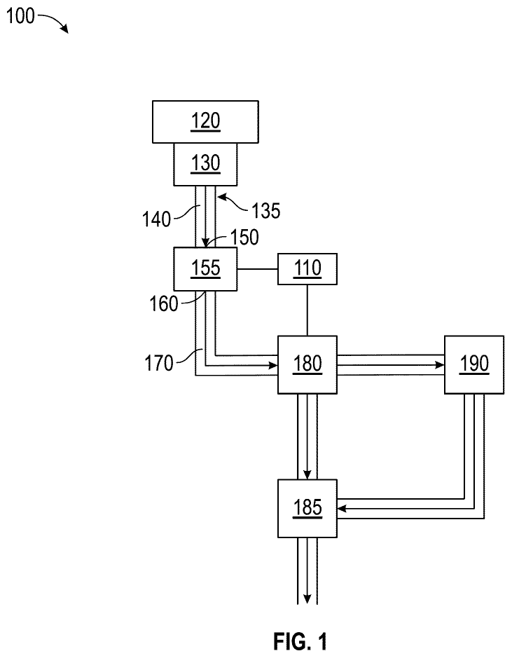

In accordance with the schematic drawing of FIG. 1, a device 100 is disclosed for continuous monitoring of volatile breathing compounds in respiratory gases, possibly of intubated patients or self-breathing patients. The device 100 can comprise a patient respiratory gas interface 120, a connector 130 coupling the patient respiratory gas interface 120 to a gas sampling line 135, and a sidestream respiratory gas monitor 155 to continuously monitor the carbon dioxide (CO₂) content of the respiratory gases. The sidestream respiratory gas monitor 155 can be a device that measures the concentration of various gases in a patient's breath (e.g., analyses the gas composition of the sampled air). The device 100 can measure the partial pressure of oxygen (pO₂), carbon dioxide (pCO₂), nitrous oxide (N₂O), and anesthetic agents, as well as the respiratory rate and end-tidal CO₂. The device can provide continuous and non-invasive monitoring of the patient's ventilation, oxygenation, and/or metabolism during anesthesia, intensive care, and/or emergency situations. The device 100 can also help detect complications such as hypoxia, hypercapnia, hypocapnia, and/or malignant hyperthermia.

The gas sampling line 135 can include a first channel 140 for conducting a predetermined flux of respiratory gases from the patient respiratory gas interface 120 to an inlet 150 of the sidestream respiratory gas monitor 155. An outlet 160 of the sidestream respiratory gas monitor 155 can be coupled to a second channel 170 for conveying the respiratory gases from the sidestream respiratory gas monitor 155. An end of the second channel 170 can be coupled to a valve 180. The valve 180 can control passage of the respiratory gas from the sidestream respiratory gas monitor 155, via the second channel 170, by connecting or disconnecting the gas flux to or from at least one volatile breathing compound detector 190. The volatile breathing compound detector 190 can measure the concentration of various organic and inorganic compounds in exhaled breath of the patient. These compounds can serve as biomarkers for various diseases, such as asthma, diabetes, lung cancer and tuberculosis. The volatile breathing compound detector 190 can utilize a sensor array and/or an electrochemical sensor that reacts with the compounds and produces electrical signals. The signals are then processed by a microcontroller and displayed on a screen or transmitted to a computer (not shown). The at least one volatile breathing compound detector 190 can be utilized for non-invasive diagnosis, monitoring, and/or screening of patients.

A pump 185 can be positioned downstream of both the sidestream respiratory gas monitor 155 and downstream of the valve 180, as well as downstream of the volatile breathing compound detector 190. This arrangement allows the pump 185 to act on the respiratory gases regardless of whether the respiratory gases are directed through the at least one volatile breathing compound detector 190 or by-passed. In some implementations, the pump 185 can be configured to generate a respiratory gas flux in the range of at least one of approximately 30-70 ml/min, or approximately 40-60 ml/min, for example, 50 ml/min. The flux through the system can support the function of the at least one volatile breathing compound detector 190. Maintaining a gas flux within these ranges can support gas exchange in the lungs, helping to ensure oxygen uptake and carbon dioxide elimination. This can be due to the alignment of the gas flow with alveolar ventilation rates—the volume of air reaching the alveoli per minute. Maintaining a gas flux within this range can also support the accuracy and reproducibility of indirect calorimetric measurements. Indirect calorimetry measures energy expenditure by analyzing respiratory gases, and its precision can depend on accurate gas sampling. By operating within the range of alveolar ventilation, the system can provide respiratory gas data that support valid energy expenditure assessments. The at least one volatile breathing compound detector 190 can continuously monitor the presence and concentration of one or more volatile breathing compounds in respiratory gases.

A momentary value of the continuously monitored CO₂ content of the respiratory gases, measured by the sidestream respiratory gas monitor 155, can be used as a signal to a control unit 110. The control unit 110 can regulate the passage of respiratory gas though the valve 180 at least based on this signal, thereby making the provision of respiratory gas to the at least one volatile breathing compound detector 190 dependent on the CO₂ content. The momentary value of CO₂ can refer to an instantaneous measurement of CO₂ concentration in the respiratory gases at a specific point during the breath cycle—for example, just as a person begins to exhale, or at the peak of exhalation when CO₂ levels are typically highest—which can be updated at a high sampling rate (e.g., several times per second) to track changes within each breath cycle. Thus, the momentary CO₂ value can be used as a signal (e.g., a real-time signal) input to a control unit 110, which can control the operation of the valve 180 upstream of the at least one volatile breathing compound detector 190. The control unit 110 dynamically regulates the opening and closing of the valve 180 in response to the changing CO₂ concentration.

The momentary values can also be considered on a breath-by-breath basis, in which the control unit 110 can use these momentary CO₂ values to determine when to direct respiratory gas to the at least one volatile breathing compound detector 190. For instance, the control unit 110 can monitor the CO₂ signal measured by the sidestream respiratory gas monitor 155 and can detect when a threshold level is reached, such as the point during exhalation when CO₂ levels rise above a certain concentration. Once this threshold is reached, the control unit 110 can open the valve 180 to allow that portion of the exhaled gas to flow to the at least one volatile breathing compound detector 190. After the relevant portion of the breath has passed, the valve 180 can be closed to prevent less meaningful portions of the respiratory cycle, such as early exhalation or inhalation containing mostly ambient air or air from the upper airways, from reaching the at least one volatile breathing compound detector 190. This process can be repeated with each breath, allowing the at least one volatile breathing compound detector 190 to capture only the most relevant part of the respiratory gas on a breath-by-breath basis, guided by the most current CO₂ measurements.

For example, in some implementations, the control unit 110 and sidestream respiratory gas monitor 155 can further monitor a CO₂ controlled signal indicating that 70% to 90% of a patient's breath is exhaled. In response, the control unit 110 causes the valve 180 to allow respiratory gases from the second channel 170 to enter into an area of the at least one volatile breathing compound detector 190 (e.g., a CO detector and/or a NO detector). When the CO₂-controlled signal indicates that all of a patient's breath is exhaled, the control unit 110 can generate an actuation signal to the valve 180, thereby preventing respiratory gases from the second channel 170 from entering an area of the at least one volatile breathing compound detector 190. This enables measurements by the CO detector and/or NO detector of the at least one volatile breathing compound detector 190 to be at least based on a part of the patient's exhaled breath. In some implementations, control unit 110 allows respiratory gases from second channel 170 to enter the area of volatile breathing compound detector 190 once 80% of a patient's breath is exhaled. The CO₂ controlled signal can provide valuable information on a patient's respiratory status and can be used to guide clinical decision-making in the management of critically ill patients.

As mentioned above, measuring can be conducted on parts (e.g., relevant parts) of the patient's breath. A shorter time of breath can be sufficient for obtaining a measurable result (e.g., an acceptable measurable result). This supports use by patients with mild to sever conditions or constraints (e.g., physiological constraints) that limit their ability to exhale for a relatively extended period, e.g., for periods of approximately ten seconds or more. Based on the construction and function of the device 100, a short exhalation period, such as a few seconds, can be enough or sufficient enough. For patients who are able to exhale for longer periods, the device 100 can still be used. In those cases, measurements can be taken over exhalations of approximately ten seconds or longer.

The use of the momentary value of the continuously monitored CO₂ content of respiratory gases as an input signal to a control unit has several benefits. For example, the use of the momentary value allows for real-time monitoring of the CO₂ content in respiratory gases, which can be utilized to control the passage of respiratory gas through the valve 180 depending on the signal. This also allows for selective sampling of respiratory gas that is most likely to contain meaningful concentrations of volatile biomarkers. The device 100 can thereby avoid analyzing respiratory gas that may be diluted with ambient air or dead space ventilation, which can contain little or no CO₂ and may not be representative of the gas composition. This passage control can therefore be used to regulate the provision of respiratory gas to at least one volatile breathing compound detector (e.g., the at least one volatile breathing compound detector 190), which is made dependent on the CO₂ content. This control mechanism can help ensure that the volatile breathing compound detector receives a consistent and accurate supply of respiratory gas, which consequently can further improve the accuracy and reliability of the detector's measurements.

Continuous monitoring of volatile breathing compounds in the respiratory gases of patients may require a device (e.g., device 100) capable of detecting and analyzing compound concentrations in real time. Based on the structural and technical features of the device in some implementations—particularly for intubated patients—the areas mentioned below can be addressed.

In some implementations, the device 100 can be sensitive enough to detect low concentrations of volatile compounds in respiratory gases, typically in the parts-per-billion (ppb) range. The device 100 can be further configured to be highly selective for specific volatile breathing compounds of interest and should not be affected by interference from other gases or compounds. The device 100 can be configured to provide real-time measurements with sufficient response even though the electrochemical sensors used typically may have slower response time than changes in the concentration of volatile breathing compounds. Such measurements are made possible due to the disclosed arrangement.

Moreover, the disclosed arrangement, according to some implementations, can be further configured to provide accurate and precise measurements of volatile compound concentrations to enable reliable diagnosis and monitoring of patients. The device 100 can be durable and robust to withstand the harsh conditions of the intensive care unit (ICU) environment and repeated use. The device 100 can be furthermore adapted to be easy to use and interpret, with a user-friendly interface and simple calibration and maintenance procedures. Finally, the construction of the device 100 can be compatible with the respiratory gases of intubated patients and the monitoring equipment already in use in the ICU.

Overall, the device 100, according to some implementations, for continuous monitoring of volatile breathing compounds in respiratory gases of intubated patients can be sensitive, selective, and reliable to provide sufficiently accurate and timely information on the patient's metabolic state and clinical condition.

In some implementations, as mentioned above, the device 100 for continuous monitoring can with a CO detector and/or an NO detector that is significantly slower in response time (e.g., there is a greater delay between the stimulus and the corresponding detection) in relation to the patient's breathing cycles. Such CO detectors and/or the NO detectors are suitably of the detector type electrochemical sensors. Electrochemical sensors are sensor types that detect and measure the concentration of a chemical species in a sample using an electrochemical reaction. These sensors are configured with either two or three electrodes, including a working electrode, a reference electrode, and sometimes a counter electrode. The electrochemical sensors utilized, according to some implementations, can detect a wide range of chemical species, including gases, ions and organic molecules. Such sensor devices are often used in environmental monitoring, medical diagnostics, and industrial process control due to their high sensitivity, relatively low cost and case of use. However, their performance can be affected by factors such as temperature, humidity, and/or interference from other species in the sample, and they require careful calibration and maintenance to ensure accurate and reliable measurements.

In some implementations, the CO detectors and/or the NO detectors (of the at least one volatile breathing compound detector 190) have been chosen with response rates of up to 90% of the response value (e.g., the maximum response value) in approximately 30 seconds. This performance set provides a good trade-off between cost and detection performance of the components. The CO₂ controlled signal that indicates that 70-90% of a patient's breath is exhaled can be at least based on the principle that exhaled breath contains a higher concentration of carbon dioxide than inhaled breath. The actuating signal, which is generated and provides input for controlling the valve 180 of the system (e.g., device 100), can then generated by a capnography monitor, which measures the concentration of CO₂ in the exhaled breath and displays it as a waveform.

As the patient exhales, the CO₂ concentration in the exhaled breath gradually increases until it reaches a peak, which corresponds to the end-tidal CO₂ (ETCO₂) concentration. The ETCO₂ can represent the average concentration of CO₂ in the alveolar gas at the end of expiration and can be a reliable indicator of the adequacy of ventilation. In healthy individuals, the ETCO₂ concentration can be typically around 35-45 mmHg. However, in critically ill patients or patients who are suffering from respiratory disease, the ETCO₂ may be outside this range, indicating a problem with ventilation or perfusion. The 70% to 90% threshold mentioned above indicates that at least 70% to 90% of the exhaled breath is being sampled, which can be considered adequate to provide an accurate measurement of the ETCO₂ concentration. This threshold can be achieved by ensuring that the capnography sampling device is placed in a location that captures exhaled breath from the functional alveoli and avoids contamination from dead space ventilation.

In the context of volatile breathing compounds, dead space can refer to the anatomical portion of the respiratory system that does not participate in gas exchange with the blood. This includes the trachea, bronchi and bronchioles, which are portions of the respiratory system that conduct air to and from the alveoli where gas exchange occurs. The presence of dead space can affect the measurement of volatile breathing compounds, as the exhaled breath from the dead space may contain a different composition of gases than the exhaled breath from the functional alveoli. This can result in a dilution effect and reduce the concentration of volatile compounds in the exhaled breath. Therefore, it can be important to consider dead space when interpreting the results of volatile compound measurements and to ensure that the sampling method captures exhaled breath from the functional alveoli. The dead space can be further divided into two types: anatomical and physiological. Anatomical dead space refers to the volume of air in the conducting airways, which can be approximately 150 ml in an average adult. Physiological dead space includes both the anatomical dead space and any additional non-functional alveoli, which can increase due to lung disease or other factors.

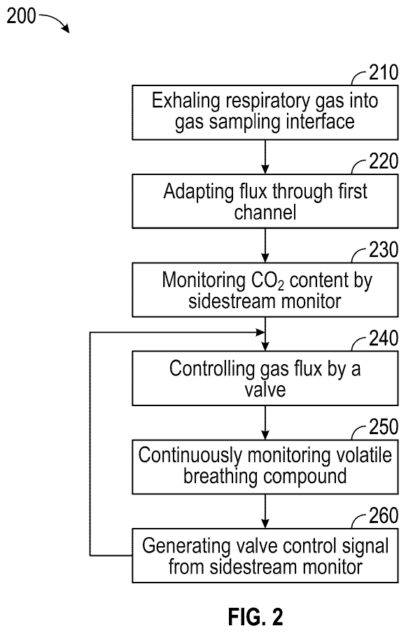

FIG. 2 illustrates an example process 200 for continuously monitoring a concentration of at least one volatile breathing compound in respiratory gases by the device 100, according to some implementations. At block 210, a respiratory gas can be exhaled into the patient respiratory gas interface 120. At block 220, the respiratory gas can enter the first channel 140 via the sampling line 135, which is coupled to the patient respiratory gas interface 120 by the connector 130. The first channel 140 can be configured to adjust the flow rate (e.g., flux) of the exhaled gas through the first channel 140.

At block 230, a portion of the exhaled gas can be diverted to the sidestream respiratory gas monitor 155. The sidestream respiratory gas monitor 155 can continuously monitor the carbon dioxide (CO₂) content of the respiratory gases. The pump 185 can facilitate a continuous flux of the respiratory gases through the sidestream respiratory gas monitor 155.

At block 240 and block 250, the valve 180 can control passage of the respiratory gas from the sidestream respiratory gas monitor 155 by connecting or disconnecting the gas flux to or from the at least one volatile breathing compound detector 190, which continuously monitors volatile breathing compound content of the respiratory gases. At block 260, a control unit 110 uses a value of the monitored CO₂ content from the at least one volatile breathing compound detector 190 to generate a signal for the valve 180, and controls the passage of respiratory gas through the valve 180 based on the signal.

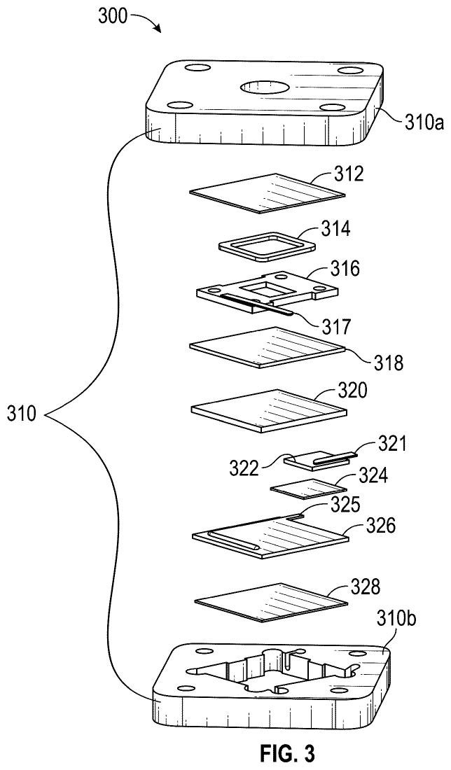

FIG. 3 illustrates an example miniaturized electrochemical sensor 300 for detection of a component in a gas. The component in the gas can be a gaseous component, such as CO. The sensor 300 can be an amperometric electrochemical sensor and can comprise a counter electrode 326 and a working electrode 316. In some implementations, the electrochemical sensor 300 also comprises a reference electrode 322. The reference electrode 322 can be positioned between the working electrode and the counter electrode and in electrical contact with an electrolyte. The reference electrode 322 can provide a constant and defined potential. This potential is can be determined by an electrolyte inside the reference electrode 322 and the reference element used. The reference electrode 322 and the counter electrode 326 can be supported by a casing 310 (e.g., having an upper part 310a and a lower part 310b) and/or substrate formed in a rigid or semi-rigid material such as plastic (e.g., polycarbonate or similar polymer compound materials), glass, ceramic, and/or silicon.

The casing 310 can comprise an upper part 310a and a lower part 310b, forming a chamber in which the sensor components and an electrolyte (e.g., a liquid electrolyte) are contained. In some implementations, the casing 310 (e.g., a rigid material casing) can be formed by sandwiching at least two slab formed structures that are designed so as to, when affixed to each other with fastening means, fixedly enclose the various components of the electrochemical sensor 300 device and encapsulate the fluid electrolyte. The rigid material casing can be 3D-printed to allow the casing 310 to be manufactured according to any suitable shape and any suitable material. The liquid electrolyte can be for instance a mild acid solution dissolved in water, such as for instance 10% H2SO4 (aq).

The electrodes can be provided with electrical connection wires 317, 321 and 325 for providing electrical contact through the casing 310. At least one of the connection wires 317, 321 and 325 connected to an electrode can be comprised from at least one of gold, palladium, ruthenium, and/or platinum. The surface of the electrodes that faces the electrolyte, at least for one of the working electrodes 316, counter electrodes 326, and reference electrodes 322, can be coated and/or covered by carbon. Additionally, the material of the working electrode 316 can comprise at least one of gold, palladium, ruthenium, and/or platinum coated with, as mentioned, with an exception for the surface facing the electrolyte. The gold, palladium, ruthenium, and/or platinum-coated portion of the working electrode 316 can be exposed to the gas. From a functional perspective, when a voltage is applied between the working electrode 316 and reference electrode 322 in an electrochemical sensor (e.g., the device 100 and/or electrochemical sensor 300), an electrochemical reaction occurs at the surface of the working electrode, which generates a current proportional to the concentration of the target species. The current is then measured and used to calculate the concentration of the target species in the sample.

The working electrode 316 can be built of two layers. On top there is a silicon sheet covered with an atomic layer deposited (ALD) platinum layer. This top layer can be for optimized and evenly distributed electron flow, with a large area connecting to both gas and the electrolyte. In close contact beneath the top layer can be a second layer with carbon content. The carbon can be for the oxidation of CO gas. The 4.0 mg/cm² platinum on carbon paper electrode can be a high performance, high platinum loading Gas Diffusion Electrode (GDE).

With respect to the counter electrode 326, the same carbon paper material 4.0 mg/cm² can be used as for the working electrode 316. Platinum can be provided also for the counter electrode 326, just as in the case of the second layer of the working electrode 316. The reference electrode 322 can be built of ELAT, a woven carbon cloth gas diffusion layer (GDL) with the particular designation LT1400. The reference electrode 322 can define the voltage relative the working electrode 316, and the choice of material allows for a broader range of materials than for the case 310 of the working electrode 316 and counter electrode 326.

With further reference to FIG. 3, the miniaturized electrochemical sensor 300 can be comprised of, in a direction from the top to the bottom, an upper part 310a of the casing 310 followed by a filter 312. The filter 312 can be a microporous polytetrafluoroethylene (PTFE) film, such as Zitex G-110, which acts to prevent passage of liquid solution below their initiation pressures, while allowing free passage of gases. The filter 312 can also protect against dehydration of the electrochemical sensor 300 as the absorbent material of the filter 312 acts as a retainer of moisture. The surfaces that face the electrolyte of at least one of the working electrodes 316, counter electrodes 326, and/or reference electrodes 322 can also be divided by filters. The filters can be comprised of Zitex.

A sealing gasket 314 can be provided between the filter 312 and the following working electrode 316. The working electrode 316 can be made using silicon wafer, which is available from for instance the manufacturer Siegert Wafer GmbH, having an atomic layer deposited (ALD) layer of platinum. Additionally, the working electrode 316 can comprise at least one of platinum, gold, palladium, carbon, and/or ruthenium. A carbon electrode 318 can be provided underneath the working electrode 316. Attached to the working electrode 316, as well as to the other electrodes, is a platinum wire 317. The wire 317 can have a diameter of between approximately 0.01 mm and 0.30 mm, for example, approximately 0.15 mm.

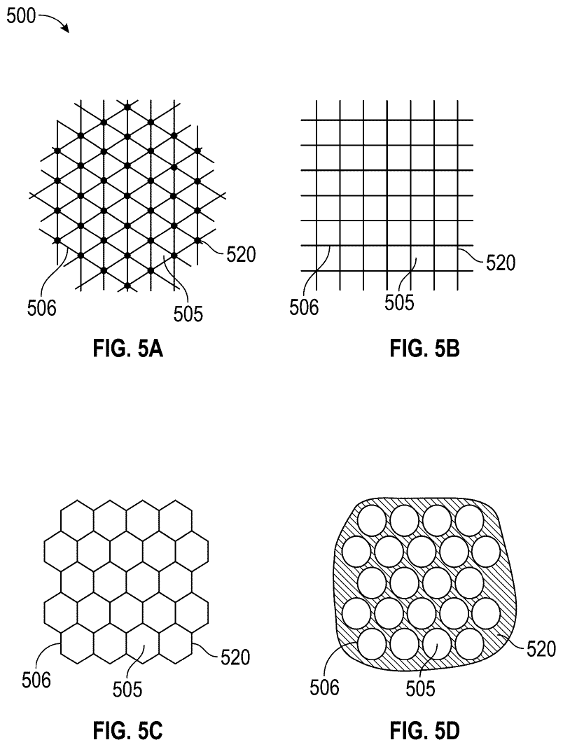

Underneath the carbon electrode 318 can be a layer 320 of porous polymeric material. The layer 320 can include a permeable electrolyte-absorbing material positioned between the working electrode 316 and the counter electrode 326. The structure of the permeable electrolyte-absorbing material can be porous, with passages formed as pores extending between the working electrode 316 and the counter electrode 326. These pores can extend in parallel throughout the layer 320. The layer 320 can have passages (see passages 505 shown in FIGS. 5A-5D) that allow transport of ions through the liquid electrolyte. The electrolyte-absorbing material can increase the surface area of the layer 320. This material can retain and deliver the electrolyte used in the electrochemical system (e.g., the electrochemical sensor 300) to support the chemical reaction. Such materials can also be used for reservoirs, general wicking, and, as in this application, for the support of fluids within electronics and sensors. In some implementations, the layer 320 can include a POREX® material. Depending on its structure, the layer of POREX can be in electrical contact with the counter electrode 326 and reference electrode 322 through the liquid electrolyte.

The reference electrode 322 can comprise a woven carbon cloth material called ELAT, the material manufactured and brought to the market by NuVant. Underneath the reference electrode is another filter 324. The filter 324 can be comprised of a microporous polytetrafluoroethylene (PTFE) film named Zitex G-110, which just as the previously described 312 filter acts to prevent passage of liquid solution below their initiation pressures, while allowing free passage of gases. The difference between the first filter 312 and the second filter 324 is the size, which is arranged to match its respective electrodes. The three electrodes 316, 322, 326 can be assembled as a sandwich with thin layers with working electrode 316, reference electrode 322, and/or counter electrode 326 on top of each other. In some implementations, the size of the working electrode 316 is about 10 mm by 10 mm. In some implementations, the size of the reference electrode 322 is about 3 mm by 3 mm. In some implementations, the size of the counter electrode is about 10 mm by 10 mm. The reference electrode 322 can act as a catalyst that ensures that the electrochemical sensor 300 is selective, e.g., specifically sensitive to certain gases.

Still in a direction from top to bottom, the second filter 324 can be followed by the counter electrode 326, a filter 328, comprised of a microporous polytetrafluoroethylene (PTFE) film named Zitex G-110, and the lower part 310b of the casing 310, which can be designed to match the upper part 310a of the casing 310. Connected to the reference electrode 322 and to the counter electrode 326 can be wires 321 and 325, respectively, which wires can be comprised from platinum in a diameter between approximately 0.01 mm and 0.30 mm or 0.15 mm, similar to the previously described wiring of the working electrode 316.

The three electrodes (e.g., working electrode 316, reference electrode 322, and/or counter electrode 326) can be, as previously described, ionically connected with the electrolyte. The electrolyte can be, also as mentioned, a mild acid solution dissolved in water such as a mild sulfuric acid of 5M concentration. A hole, such as a hole of 4 mm diameter, can be provided for access of gas, e.g., carbon monoxide, to the working electrode 316. The casing 310 can be made of the plastic material PEEK, which is milled to a depth, such as a depth of approximately 2 mm, for the sensor to fit. By PEEK is meant polyether ether ketone, which is an originally colorless organic thermoplastic polymer in the polyaryletherketone (PAEK) family of materials, used in a variety of engineering applications. A hole can also be provided at the counter electrode 326 for gas access. The total thickness of the assembled electrochemical sensor 300 can be between approximately 1 mm and 10 mm or between approximately 2 mm and 3 mm. The casing top (e.g., the upper part 310a) and bottom (e.g., the lower part 310b) can be pressed together slightly in order to compress the more flexible materials in the sandwich structure.

The reference voltage can be set to +0.30 V to optimize the sensor for carbon monoxide detection and allows for increased sensitivity. It is possible also with higher voltages up to about +0.7 V, but then with a risk of a possible disadvantage associated with potentially enhanced interference of other gases.

With respect to utilization of carbon paper material with platinum, the reasonable amount of loading of platinum of approximately 4 mg/cm² ensures that there is sufficient platinum for ensuring high active surface area to achieve strong response to low concentrations of carbon monoxide. With superior catalytic activity against carbon monoxide, a high loading increases the number of active sites available for CO oxidation, thereby enhancing the sensor sensitivity.

The CO sensor can have a current response that is linear to the concentration. This is described by Fick's laws of diffusion, where Fick's first law describes that movement of particles from high to low concentration (e.g., diffusive flux), which is directly proportional to the particle's concentration gradient.

In view of the above, an adequate amount of platinum loading can ensure that there is a sufficient response to the concentration. Platinum also does not corrode at higher concentrations of acid. Therefore, the material is stable and ensures long-term stability of the sensor. Carbon materials are rugged, stable, and/or chemically resistant to higher concentrations of acid. Carbon materials such as nanotube, graphite, carbon powder is readily available. High electrical conductivity and low cost are further attractive properties of the carbon materials for this application.

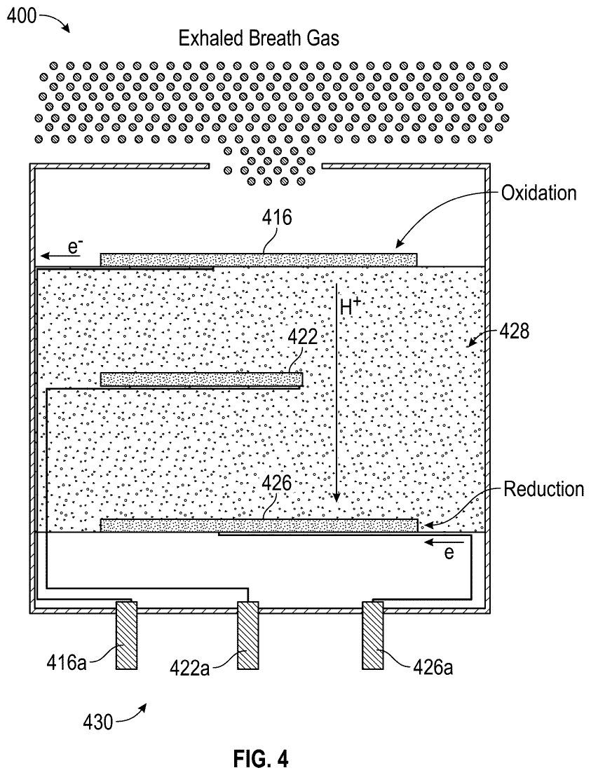

FIG. 4 shows an electrochemical cell 400 in operation. The working principle of an electrochemical carbon monoxide sensor can include three electrodes immersed in a liquid electrolyte 428. The first electrode, e.g., the working electrode 416, can comprise platinum and the function of the working electrode 416 is to catalyze the oxidation of CO to CO₂. It is backed by a gas-permeable, but hydrophobic, membrane, that allows CO gas to diffuse through. The electrochemical reaction at the working electrode 416 produces electrons, which flow through the external circuit, generating the sensor's output signal. The second electrode, e.g., the reference electrode 422, can provide a stable electrochemical potential in the electrolyte 428. It remains constant and is protected from exposure to CO gas. The third electrode, e.g., the counter electrode 426, can complete the circuit in that the counter electrode 426 allows electrons to enter or leave the electrolyte 428.

The electrochemical cell's 400 potentiostat 430 can control the working electrode potential, convert the signal current to a voltage, and/or maintain a constant voltage between the reference electrode 422 and working electrode 416. The working electrode 416 can be connected to a working terminal 416a of the potentiostat 430. The reference electrode 422 can be coupled to a reference terminal 422a, which provides a stable and well-defined potential against which the potential of the working electrode 416 is measured. The reference electrode 422 can carry minimal to no current, ensuring its potential remains constant during operation. The counter electrode 426 can be coupled to a counter terminal 426a of the potentiostat 430 and functions to complete the electrical circuit by sourcing or sinking current as needed to maintain the desired potential at the working electrode 426. A chemically selective filter can remove interfering gases before they reach the working electrode 416. The electrochemical cell 400 can detect CO by measuring the electrochemical reaction within the sensor, creating an electrical output proportional to the CO level.

Oxidation and reduction in the electrochemical cell 400 during operation occurs according to the following. Oxidation at the working electrode 416 occurs in that carbon monoxide (CO) gas diffuses through the gas-permeable membrane to reach the working electrode 416. At the working electrode 416, CO undergoes oxidation:

CO+H₂O→CO₂+2e⁻

The reaction produces electrons (2e⁻), which flow through the external circuit. Reduction at the counter electrode 426 occurs in that electrons from the working electrode 416 travel through the external circuit and reach the counter electrode 426. At the counter electrode 426, reduction occurs:

O₂+4e⁻+2H₂O→4OH⁻

This balances the charge and completes the electrochemical circuit. The reference electrode 422 maintains a stable electrochemical potential, serving as a reference point. It remains unaffected by CO exposure. As previously mentioned, the potentiostat controls the working electrode potential, which can ensure a constant voltage between the reference electrode 422 and the working electrode 416. The resulting current (due to the oxidation of CO) can be converted to a voltage signal, providing a measure of CO concentration.

FIGS. 5A-5D illustrate an electrochemical sensor 500, according to some implementations, in which the various layers have been dismantled from each other for improved visibility. With reference to FIGS. 5A-5D, the layer 520 comprising a POREX structure in some implementations, can define passages 505 delimited by the walls 506, is, shown in further detail. The passages 505 can be straight pores arranged in parallel and distributed over the layer 520. The layer 520 can support a working electrode (e.g., the working electrode 316 and/or working electrode 416), which covers the walls 506 of the layer 520 and thus extends along the passages 505 from each respective side of the layer 520.

The portion of the layer 520 can be provided with passages 505 which can cover a defined area of some mm², such as 6 by 6 mm². The passages 505 can have cross-sectional dimensions in the range of approximately 1-300 micrometers, or in the range of approximately 10-150 micrometers, for example, about 120 micrometers. The width of the walls 506 of the grid defining the passages 505 can be in the range of approximately 1-100 micrometers, for example, about 20 micrometers. The length of the passages 505 can be in the range of approximately 10-2,000 micrometers, or in the rage of approximately 50-850 micrometers, for example, about 300 micrometers. The term aspect ratio (AR) is defined as a ratio of height (h) to width (w) of a structure or passage, e.g., AR=h/w. Thus, the aspect ratio AR of the passages 505 can be at least 0.25, at least 1, at least 4, at least 10, at least 20, and/or at least 50. A high AR can provide a large surface area of the walls 506 defining the passages 505 in the layer 520.

In FIGS. 5A-5D, four different arrangements of passages 505 and walls 506 are disclosed. FIG. 5A shows an arrangement of passages 505 having a triangular cross-section in the layer 520. The passages 505 can be defined by walls 506 arranged at angles of 60 degrees with respect to reach other. Thus, the passage 505 of FIG. 5A can be defined by three walls 506 extending along the passage. FIG. 5B shows an arrangement of passages 505 having a hexagonal cross-section in the layer 520. The passages 505 can be defined by six walls 506 arranged at angles of 120 degrees with respect to reach other. FIG. 5C shows a quadratic arrangement of passages 505 in the layer 520. The passages 505 shown in FIG. 5C can be defined by walls 506 arranged at right angles with respect to reach other. Thus, the passage 505 can be defined by four walls 506 extending along the passage 505. Lastly, FIG. 5D shows an arrangement of cylindrical passages 505 formed in the layer 520. In this case the passage 505 of FIG. 5D can be defined by walls 506 forming segments of the cylindrical passage extending through the layer 520. In each of these examples, the passages 505 can be closely arranged, forming a close packed arrangement of passages 505 in the layer 520.

In the following, a non-limiting example of manufacturing a miniaturized electrochemical sensor (e.g., the electrochemical sensor 300 and/or the electrochemical cell 400) is disclosed. The structure supporting the working electrode (e.g., the working electrode 316 and/or the working electrode 416) can be manufactured by providing a double side polished 100 mm diameter, 300 micrometers thick silicon wafer. The silicon wafer can be spin coated with a 6 micrometers thick layer of a photoresist (e.g., AZ 9260). The wafer with the photoresist layer can then be soft baked on a hot plate for 2 minutes and thereafter exposed with UV light at an intensity of 300 mW/cm² for 15 seconds, through a lithography mask. The photoresist can then be developed using developer 2401 for 3 minutes in order to define a pattern. The pattern can define the walls and passages of the structure 4. The structure can be etched using deep reactive ion etching for 1.5 hours to form a grid structure having walls and passages.

The etched silicon wafer can be transferred to an atomic layer deposition chamber (Beneq TFS 200). Here, a 10 nm layer of Al₂O₃ can deposited on to the structure followed by a 10 nm thick platinum layer. The wafer can be diced into chips of dimensions about 10×10 mm².

The manufacturing of the counter and reference electrode can be carried out on a 2 mm thick polycarbonate (PC) substrate. Silver of thickness 500 nm can be deposited on one side of the PC substrate using e-beam evaporation. The silver can be patterned to define counter and reference electrode. The reference electrode is oxidized to AgO₂ by applying a voltage of 1.0 V to the silver electrode, which is the anode by using a platinum electrode as the cathode.

The chip with the working electrode (e.g., the working electrode 316 and/or the working electrode 416) can be fastened, e.g., glued, on top of the counter electrode (e.g., the counter electrode 326 and/or the counter electrode 426) and/or the reference electrode (e.g., the reference electrode 322 and/or the reference electrode 422). The assembly can thereafter be submerged into a liquid electrolyte solution and put in a vacuum desiccator to fill the chamber between the working and the counter/reference electrode. The liquid electrolyte can ionically, thus electrically, connect the working, counter, and/or the reference electrodes.

During operation of the electrochemical sensor, a gas to be analyzed can provided at the first surface of the sensor. The potential at the working electrode can be kept at +0.7 V with respect to the Ag/AgO₂ reference electrode.

The counter electrode can enable a current to flow through the sensor cell. The working potential, electrolyte, and/or electrode materials can be selected so that the gas being measured is oxidized at the working electrode. The layer (e.g., a POREX layer) can act as a diffusion layer that permits an interaction between the gas, electrode and liquid. As the oxidization takes place at the working electrode, oxygen can be reduced to water at the counter electrode. The resulting current, which flows through the sensor, can be directly proportional to the gas concentration. Thus, the oxidation of the analyte, in this case CO, at the working electrode can result in a current that is detected by using a potentiostat that comprises a transimpedance amplifier. It is also used for maintaining a constant potential between the working and the reference electrode.

The manufactured sensor has been tested to characterize levels of 0 to 100 ppb of CO in exhaled breath. The detection limit (S/N=2) was estimated to be 0.3 ppb and the sensitivity was measured to be 4 microA/ppm/cm². The response and the recovery time of the sensor (time to return to 90% of starting signal) were measured to be 6 seconds.

The person skilled in the art realizes that the present disclosure is not limited to the implementations described above. The person skilled in the art further realizes that modifications and variations are possible within the scope of the appended claims. Additionally, variations to the disclosed implementations can be understood and effected by the skilled person in practicing the claimed disclosure, from studying the drawings, the disclosure and the appended claims.

Unless the context clearly requires otherwise, throughout the description and the claims, the words “comprise,” “comprising,” “include,” “including” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Likewise, the word “connected”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number, respectively. The word “or” in reference to a list of two or more items, that word covers all of the following interpretations of the word: any of the items in the list, all of the items in the list, and any combination of the items in the list.

Moreover, conditional language used herein, such as, among others, “can,” “could,” “might,” “may,” “e.g.,” “for example,” “such as” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments.

While certain embodiments have been described, these embodiments have been presented by way of example only, and are not intended to limit the scope of the disclosure. For example, while illustrated embodiments include preparation for direct hybrid bonding, the skilled artisan will appreciate that the techniques taught herein can be useful for direct metal bonding even in the absence of direct dielectric bonding. Indeed, the novel apparatus, methods, and systems described herein may be embodied in a variety of other forms; furthermore, various omissions, substitutions and changes in the form of the methods and systems described herein may be made without departing from the spirit of the disclosure. For example, while blocks are presented in a given arrangement, alternative embodiments may perform similar functionalities with different components and/or circuit topologies, and some blocks may be deleted, moved, added, subdivided, combined, and/or modified. Each of these blocks may be implemented in a variety of different ways. Any suitable combination of the elements and acts of the various embodiments described above can be combined to provide further embodiments. It is also contemplated that various combinations or sub-combinations of the specific features and aspects of the embodiments may be made and still fall within the scope. The accompanying claims and their equivalents are intended to cover such forms or modifications as would fall within the scope and spirit of the disclosure.

What is claimed is:

1. A device for continuous monitoring of a concentration of at least one volatile breathing compound in respiratory gases of patients, the device comprising:
a patient respiratory gas interface;
a connector adapted to couple the patient respiratory gas interface to a gas sampling line, the gas sampling line comprising a first channel for conducting a predetermined flux of the respiratory gases from the patient respiratory gas interface to an inlet of a sidestream respiratory gas monitor, wherein the sidestream respiratory gas monitor is adapted to continuously monitor carbon dioxide (CO₂) content of the respiratory gases;
a pump arranged to facilitate a continuous flux of the respiratory gases through the device including through the sidestream respiratory gas monitor; and
an outlet of the sidestream respiratory gas monitor coupled to a second channel of the gas sampling line for conveying the respiratory gases from the sidestream respiratory gas monitor, the second channel coupled to a valve, wherein the valve is adapted to control passage of the respiratory gas from the sidestream respiratory gas monitor via the second channel by connecting or disconnecting the gas flux to or from at least one volatile breathing compound detector, wherein the at least one volatile breathing compound detector is adapted to continuously monitor volatile breathing compound content of the respiratory gases led to the at least one breathing compound via the valve;
wherein a momentary value of the continuously monitored CO₂ content of the respiratory gases is monitored by a control unit, wherein the control unit is configured to transmit a signal to the valve based on the momentary value, wherein the passage of the respiratory gas though the valve is controlled in dependence of the signal, wherein a provision of the respiratory gas can be controlled to be either lead through the at least one volatile breathing compound detector or by-passed in dependence on the momentary value of the CO₂ content.

2. The device of claim 1, wherein the pump is located downstream of the sidestream respiratory gas monitor and downstream of the valve and volatile breathing compound detector, wherein the pump acts on the respiratory gases irrespective of whether the respiratory gases are led through the at least one volatile breathing compound detector or by-passed.

3. The device of claim 1, wherein the device is adapted to function in a manner such that a CO₂ controlled signal indicating that 70% to 90% of a patient's breath is exhaled causes the control unit controls to control the valve to allow the respiratory gases from the second channel to enter into an area of the at least one volatile breathing compound detector, and that the CO₂ controlled signal indicates that a portion of or all of a patent's breath is exhaled causes the control unit control to control the valve to by-pass the respiratory gases from the second channel as exhaust gases.

4. The device of claim 3, wherein the control unit allows the respiratory gases from the second channel to enter the at least one volatile breathing compound detector once 80% of a patient's breath is exhaled.

5. The device of claim 1, wherein the at least one volatile breathing compound detector is slower in response time in relation to a patient's breathing cycles.

6. The device of claim 1, wherein the at least one volatile breathing compound detector comprises an electrochemical sensor detecting at least one of CO and NO.

7. The device of claim 1, wherein the at least one volatile breathing compound detector has a response rate of up to 90% of a response value in approximately 30 seconds.

8. The device of claim 1, wherein the device is adapted for intubated patients.

9. The device of claim 1, wherein the device is adapted for spontaneously or self-breathing patients in that particularly high concentrations of CO and/or NO are measured.

10. A miniaturized electrochemical sensor for detection of a component in a gas, the sensor comprising:
a casing comprising a rigid material;
at least two electrodes, one of which comprises a working electrode and one of which electrodes comprises a counter electrode, wherein both of the at least two electrodes are at least partly enclosed within the casing while still in contact with an environment surrounding the casing;
connection wires connected to each of the at least two electrodes;
the casing encapsulating a liquid electrolyte, wherein the liquid electrolyte is contained in-between the working electrode and the counter electrode; and
a permeable electrolyte absorbing material provided between the working electrode and the counter electrode, the permeable electrolyte absorbing material comprising a structure with a plurality of passages extending between the working electrode and the counter electrode to allow ions of the electrolyte to be transported between the working electrode and the counter electrode.

11. The sensor of claim 10, wherein the liquid electrolyte comprises sulfuric acid (H₂SO₄).

12. The sensor of claim 10, further comprising a reference electrode provided between the working electrode and the counter electrode and in electrical contact with the electrolyte.

13. The sensor of claim 10, wherein the rigid material casing is formed by sandwiching at least two slab formed structures that are configured to fixedly enclose by fasteners the miniaturized electrochemical sensor and encapsulate the liquid electrolyte.

14. The sensor of claim 10, wherein the rigid material casing is 3D-printed.

15. The sensor of claim 10, wherein surfaces that face the electrolyte of at least one of the working electrodes and the counter electrodes are divided by filters.

16. The sensor of claim 10, wherein surfaces that face the electrolyte of at least one of the working electrodes and the counter electrodes are divided by an electrolyte absorbing filter material.

17. The sensor of claim 16, wherein the structure of the electrolyte absorbing material is porous, and wherein the passages are formed as pores.

18. The sensor of claim 17, wherein the pores of the electrolyte absorbing material extend in parallel throughout the structure of the electrolyte absorbing material.

19. The sensor of claim 10, wherein a surface of the working electrode facing the gas comprises at least one of gold, palladium, ruthenium, and/or platinum coated and the surface facing the electrolyte is coated and/or covered by carbon.

20. The sensor of claim 10, wherein at least one of the connection wires connected to the at least two electrodes is comprised from at least one of gold, palladium, ruthenium, and/or platinum.
